(19) United States
(12) Patent Application Publication
(10) Pub. No.: US 2009/0215127 A1
(43) Pub. Date: Aug. 27, 2009

(54) ph Adjustment Free System For Producing Fermentable Sugars and Alcohol

(75) Inventors: Suzanne Breneman, Orfordville, WI (US); Brad A. Paulson, Brodhead, WI (US); Vivek Sharma, Beloit, WI (US); Jayarama K. Shetty, Pleasanton, CA (US)

Correspondence Address: DANISCO US INC.;ATTENTION: LEGAL DEPARTMENT, 925 PAGE MILL ROAD, PALO ALTO, CA, 94304, US

(73) Assignee: DANISCO US INC., GENENCOR DIVISION, Palo Alto, CA (US)

(21) Appl. No.: 12/366,333

(22) Filed: Feb. 5, 2009

Related U.S. Application Data
(60) Provisional application No. 61/026,510, filed on Feb. 6, 2008.

(51) Int. Cl. C12P 19/14; C08B 31/00; C12P 19/22; C12P 19/18; C12P 7/02; C12P 7/06

(52) U.S. Cl. 435/95; 536/102; 435/99; 435/97; 435/155; 435/161

(57) ABSTRACT

The present invention relates to a process for producing downstream products, such as fermentable sugars (e.g., glucose) and alcohols (e.g., ethanol) from starch-containing material (e.g., grain) without a pH adjustment before or after the starch liquefaction step.

### CROSS-REFERENCE

This application claims priority to U.S. Application Ser. No. 61/026,510 filed Feb. 6, 2008, which is incorporated herein in its entirety.

### FIELD OF THE INVENTION

The present invention relates to a process for producing downstream products, such as fermentable sugars (e.g., glucose) and alcohols (e.g., ethanol) from starch-containing material (e.g., grain) without a pH adjustment before or after the starch liquefaction step.

### BACKGROUND OF THE INVENTION

In general, starch to fermentable sugar and/or alcohol processing includes a number of steps. In a typical process, grains and cereals containing granular starch are milled. Two processes of milling are generally used and these are referred to in the art as wet milling and dry milling. Milled starch-containing material is then mixed with an aqueous solution to produce a slurry having a dry solids content ranging from 25% to 45%. Typically in a dry milling process, the aqueous solution that is mixed with the milled starch-containing material includes not only water but also varying amounts of thin stillage. The addition of thin stillage to the slurry necessitates the pH adjustment of the slurry. For example, when milled whole ground corn grain is used as a starch-containing material and mixed with water, the pH of the slurry is about pH 5.8 to about pH 6.2. However, the pH of the slurry is reduced by the addition of thin stillage to about pH 4.8 to pH 5.2. The thin stillage is used by the industry to conserve water usage in fermentable sugar and/or alcohol processing. The starch is then converted to short chain less viscous dextrins by a liquefaction process which generally involves gelatinization of the starch simultaneously with or followed by addition of alpha amylase.

The alpha amylases currently used in most commercial liquefaction processes are not stable at the pH levels of pH 4.8 to pH 5.2, and therefore the pH of the slurry is adjusted to about pH 5.6 to 6.0 using suitable alkali (e.g., sodium or calcium hydroxide, sodium carbonate or ammonia).

The liquefied starch is then converted to low molecular weight sugars by a saccharification step which typically includes enzymatically using a glucoamylase. The low molecule weight sugars may be further purified (e.g. to purified dextrose), isomerized (e.g. to fructose) or metabolized by a fermenting microorganism such as yeast (e.g. to ethanol). Frequently the saccharification and fermentation steps may be carried out simultaneously. Starting yeast fermentations at a pH of 5.6 to 6.0 can result in a high risk of microbial contamination and therefore industrial alcohol producers generally adjust the pH after liquefaction down to a pH less than 5.0 using for example dilute acid (e.g. sulfuric acid).

The pH adjustments required before and after the liquefaction step to provide appropriate conditions for liquefaction and yeast fermentation may result in high salt accumulation in the fermentation medium and a high sulphur content which may create an environmental disposal problem.

While numerous improvements have been made for the liquefaction, saccharification, and fermentation processes for producing fermentable sugars and alcohols from starch containing materials, a need still exists for more efficient means for these process steps.

### BRIEF SUMMARY OF THE INVENTION

The present invention relates to a process for the production of fermentable sugars and/or alcohol which does not require a pH adjustment and more specifically does not require a) the addition of alkali to increase the pH during the liquefaction step(s) and/or b) the addition of acid to decrease the pH for the fermentation step. Reference is made to FIG. 1.

One aspect of the invention relates to a pH adjustment free liquefaction step, wherein the pH of the liquefaction is in the range of pH 4.5 to 5.4 and acid neutralizing chemicals are not added to the liquefaction process step.

In another aspect, the invention relates to a process for producing a fermentable sugar comprising a) mixing milled starch-containing material with water and thin stillage, wherein the thin stillage is in the range of 10 to 70% v/v and obtaining a slurry comprising starch and having a dry solids (ds) content of 20 to 50% w/w, b) treating the slurry with a phytase prior to or simultaneously with liquefying the starch, c) liquefying the starch, d) adding an alpha amylase to the starch either during step b) and/or simultaneously with the liquefying step and e) saccharifying the liquefied starch to obtain fermentable sugars, wherein the pH is not adjusted during any of the steps a), b), c), d) or e). In some embodiments, the fermentable sugar is recovered and purified or isomerized. In other embodiments, the phytase is added prior to the liquefaction step. In further embodiments, the alpha amylase is added with the phytase. In yet further embodiments, a second alpha amylase dose is added during the liquefaction step.

In a further aspect, the invention relates to a process of producing alcohol from the starch-containing material comprising liquefying and saccharifying the liquefied starch as disclosed above to obtain fermentable sugars and further fermenting the fermentable sugars under suitable fermentation conditions using a fermenting microorganism to obtain alcohol. In some embodiments, the saccharification and fermentation steps are simultaneous. In some embodiments, the alcohol is ethanol. In a particular aspect the invention relates to a method of producing an alcohol from starch comprising the steps: (a) mixing starch with water and thin stillage to obtain a slurry, (b) treating the slurry with a phytase, (c) liquefying the starch, (d) adding an alpha-amylase, (e) saccharifying the liquefied starch to obtain fermentable sugars, and (f) fermenting the fermentable sugars using a fermenting microorganism to obtain an alcohol, wherein a pH adjustment is not performed during any of the steps (a), (b), (c), (d), (e), or (f).

### BRIEF DESCRIPTION OF THE DRAWINGS

FIG. 1 illustrates a process flow diagram for an embodiment of the process in the production of ethanol without a pH adjustment during the process steps.

FIG. 2 shows the effect of phytase treatment of whole ground corn on the increase in the thermostability and low pH stability of SPEZYME XTRA.

FIG. 3 shows the effect of phytase addition during primary liquefaction of whole ground corn on the viscosity reduction after jet cooking.

FIG. 4 shows a comparison of sulfate and phytic acid content in DDGS: 1) from a conventional process, and 2) from the process with no pH adjustment. The gray line is for the conventional process. The black line is for DDGS from the process with no pH adjustment, and reference is made to Example 4.

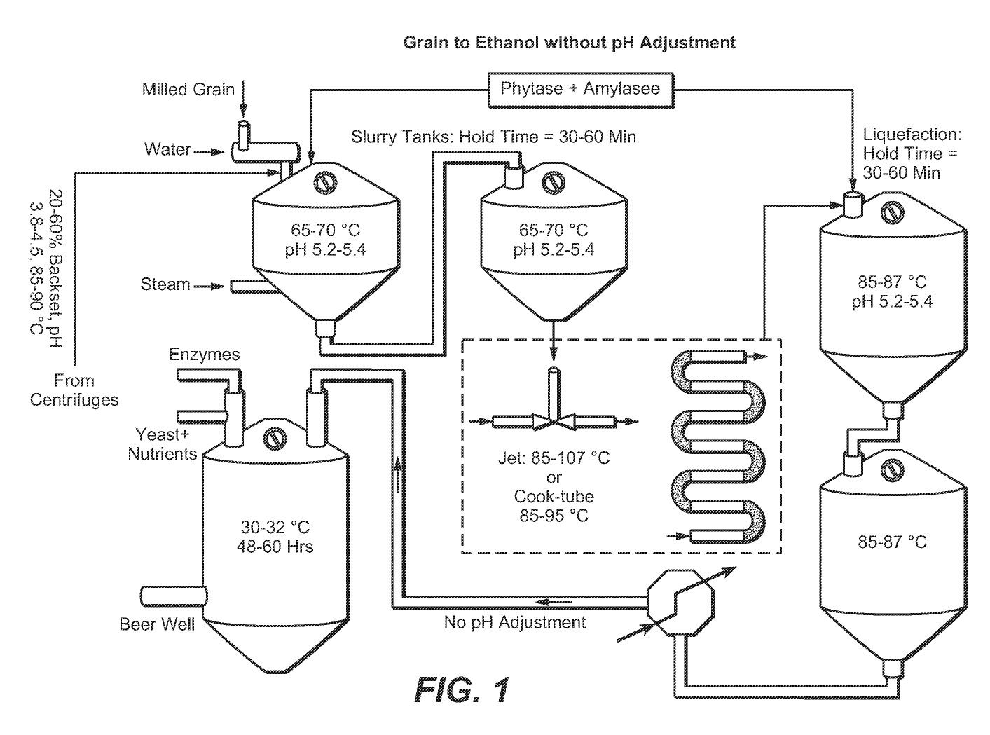

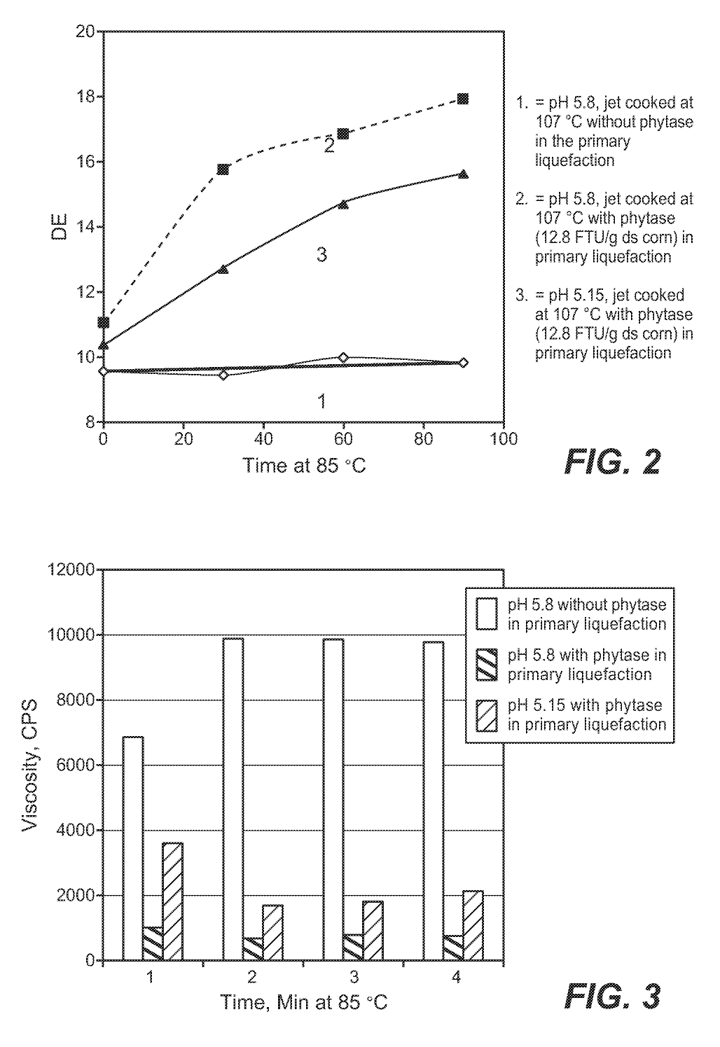

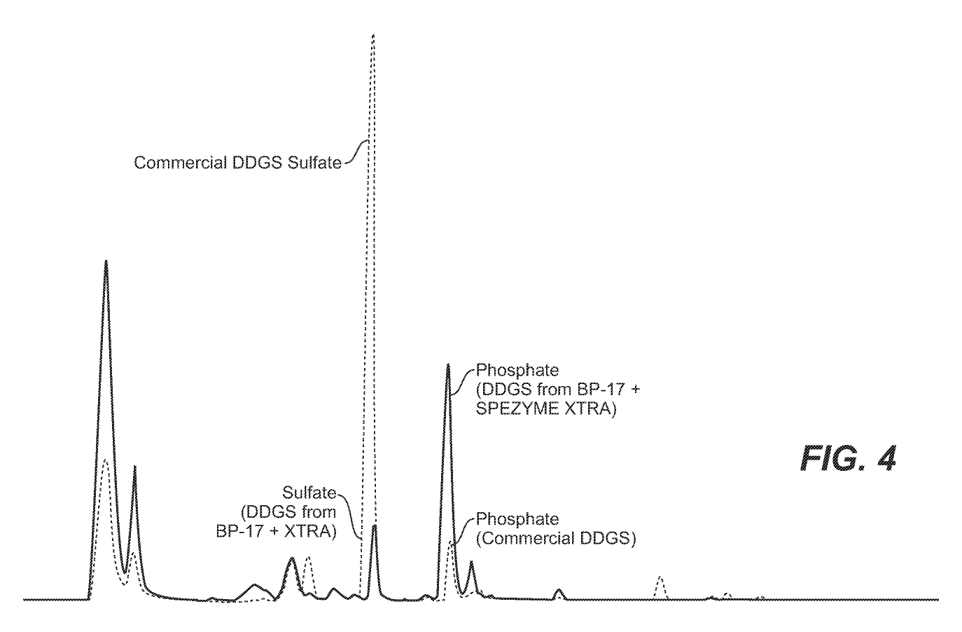

### DETAILED DESCRIPTION OF THE INVENTION

### Definitions

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Although any methods and materials similar or equivalent to those described herein can be used in the practice or testing of the present invention, the preferred methods and materials are described.

The invention will now be described in detail by way of reference only using the following definitions and examples. All patents and publications, including all sequences disclosed within such patents and publications, referred to herein are expressly incorporated by reference.

“Alpha amylases” are α-1,4-glucan-4-glucanohydrolases (E.C. 3.2.1.1) and are enzymes that cleave or hydrolyze internal α-1,4-glycosidic linkages in starch (e.g. amylopectin or amylose polymers).

“Liquefaction” or “liquefy” means a process by which starch is converted to shorter chain and less viscous dextrins.

“Dextrins” are short chain polymers of glucose (e.g., 2 to 10 units).

The term “starch” refers to any material comprised of the complex polysaccharide carbohydrates of plants, comprised of amylose and amylopectin with the formula (C₆H₁₀O₅)ₓ, wherein x can be any number.

The phrase “wherein the pH is not adjusted” or “without pH adjustment” means additional acid or alkali compounds is not added to adjust the pH at any step of the process to produce fermentable sugars and/or alcohol from milled containing starch materials.

The term “granular starch” means raw starch, that is, starch which has not been subject to temperatures of gelatinization.

The terms “saccharifying enzyme” and “glucoamylase (E.C. 3.2.1.3)” are used interchangeably herein and refer to any enzyme that is capable of catalyzing the release of D-glucose from the non-reducing ends of starch and related oligo- and polysaccharides.

The term “oligosaccharides” refers to any compound having 2 to 10 monosaccharide units joined in glycosidic linkages. These short chain polymers of simple sugars include dextrins.

The term “fermentable sugar” refers to simple sugars such as monosaccharides and disaccharides (e.g. glucose, fructose, galactose, sucrose) that can be used by a microorganism in enzymatic conversion to end-products (e.g. ethanol).

The term “DE” or “dextrose equivalent” is an industry standard for measuring the concentration of total reducing sugars, calculated as D-glucose on a dry weight basis. Unhydrolyzed granular starch has a DE that is essentially 0 and D-glucose has a DE of 100.

The term “total sugar content” refers to the total sugar content present in a starch composition.

The term “dry solids (ds)” refers to the total solids of a slurry in % on a dry weight basis.

The term “milled” is used herein to refer to starch-containing material that has been reduced in size, such as by grinding, crushing, fractionating or any other means of particle size reduction. Milling includes dry or wet milling. “Dry milling” refers to the milling of whole dry grain. “Wet milling” refers to a process whereby grain is first soaked (steeped) in water to soften the grain.

The term “gelatinization” means solubilization of a starch molecule, generally by cooking, to form a viscous suspension.

The term “gelatinization temperature” refers to the lowest temperature at which gelatinization of a starch containing substrate begins. The exact temperature of gelatinization depends on the specific starch and may vary depending on factors such as plant species and environmental and growth conditions.

The term “below the gelatinization temperature” refers to a temperature that is less than the gelatinization temperature.

The term “slurry” refers to an aqueous mixture comprising insoluble solids, (e.g. granular starch).

The term “fermentation” refers to the enzymatic and anaerobic breakdown of organic substances by microorganisms to produce simpler organic compounds. While fermentation occurs under anaerobic conditions it is not intended that the term be solely limited to strict anaerobic conditions, as fermentation also occurs in the presence of oxygen.

The phrase “simultaneous saccharification and fermentation (SSF)” refers to a process in the production of end products in which a fermenting organism, such as an ethanol producing microorganism, and at least one enzyme, such as a saccharifying enzyme are combined in the same process step in the same vessel.

The term “thin stillage” means the liquid portion of stillage separated from the solids (e.g., by screening or centrifugation) which contains suspended fine particles and dissolved material.

The term “backset” is used to mean recycled thin stillage.

The term “Distillers feeds” means the by-products of fermentation of cereal grains and includes Distillers dried grain with solubles (DDGS) and/or Distillers dried grain (DDG).

The term “end product” refers to any carbon-source derived product which is enzymatically converted from a fermentable substrate. In some preferred embodiments, the end product is an alcohol (e.g., ethanol).

The term “derived” encompasses the terms “originated from”, “obtained” or “obtainable from”, and “isolated from” and in some embodiments as used herein means that a polypeptide encoded by the nucleotide sequence is produced from a cell in which the nucleotide is naturally present or in which the nucleotide has been inserted.

As used herein the term “fermenting organism” refers to any microorganism or cell, which is suitable for use in fermentation for directly or indirectly producing an end product.

The terms “recovered”, “isolated”, and “separated” as used herein refer to a protein, cell, nucleic acid or amino acid that is removed from at least one component with which it is naturally associated.

The terms “protein” and “polypeptide” are used interchangeability herein. In the present disclosure and claims, the conventional one-letter and three-letter codes for amino acid residues are used. The 3-letter code for amino acids as defined in conformity with the IUPAC-IUB Joint Commission on Biochemical Nomenclature (JCBN). It is also understood that a polypeptide may be coded for by more than one nucleotide sequence due to the degeneracy of the genetic code.

As used herein, the term “phytase” refers to an enzyme which is capable of catalyzing the hydrolysis of esters of phosphoric acid, including phytate and releasing inorganic phosphate and inositol. In some embodiments, in addition to phytate, the phytase may be capable of hydrolyzing at least one of the inositol-phosphates of intermediate degrees of phosphorylation.

The terms, “thermostability” and “thermal stability” are used interchangeably and mean heat stability.

The term “pH stability” means stability of an enzyme at a given pH.

The phrase “phytic acid inhibition” means loss of alpha amylase activity due to high levels of phytic acid. “IP6” is defined as inositol containing 6 phosphate groups. IP6 is usually found with various amounts of its derivatives each having 1 to 5 phosphate groups (IP5-IP1).

Although any methods and materials similar or equivalent to those described herein can be used in the practice or testing of the present invention, exemplary and preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a cell” includes reference to one or more cells and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Other definitions of terms may appear throughout the specification. Before the exemplary embodiments are described in more detail, it is to be understood that this invention is not limited to particular embodiments described, as such may, of course, vary.

The publications discussed herein are provided solely for their disclosure prior to the filing date of the present application. Nothing herein is to be construed as an admission that the present invention is not entitled to antedate such publication by virtue of prior invention.

### Starch-Containing Material

Starch-containing materials useful according to the invention include any starch-containing material. Preferred starch-containing material may be obtained from wheat, corn, rye, sorghum (milo), rice, millet, barley, triticale, cassaya (tapioca), potato, sweet potato, sugar beets, sugarcane, and legumes such as soybean and peas. Preferred material includes corn, barley, wheat, rice, milo and combinations thereof. Plant material may include hybrid varieties and genetically modified varieties (e.g. transgenic corn, barley or soybeans comprising heterologous genes). Any part of the plant may be used as a starch-containing material, including but not limited to, plant parts such as leaves, stems, hulls, husks, tubers, cobs, grains and the like. In some embodiments, essentially the entire plant may be used, for example, the entire corn stover may be used. In some embodiments, whole grain may be used as a starch-containing material. Preferred whole grains include corn, wheat, rye, barley, sorghum and combinations thereof. In other embodiments, starch-containing material may be obtained from fractionated cereal grains including fiber, endosperm and/or germ components. Methods for fractionating plant material, such as corn and wheat, are known in the art. In some embodiments, starch-containing material obtained from different sources may be mixed together to obtain material used in the processes of the invention (e.g. corn and milo or corn and barley).

### Milling Starch-Containing Material

In some embodiments, starch-containing material may be prepared by means such as milling. Two general milling processes include wet milling or dry milling. In dry milling for example, the whole grain is milled and used in the process. In wet milling the grain is separated (e.g. the germ from the meal). In particular, means of milling whole cereal grains are well known and include the use of hammer mills and roller mills. Methods of milling are well known in the art and reference is made TO THE ALCOHOL TEXTBOOK: A REFERENCE FOR THE BEVERAGE, FUEL AND INDUSTRIAL ALCOHOL INDUSTRIES 3 ED. K. A. Jacques et al., Eds, (1999) Nottingham University Press. See, Chapters 2 and 4. In some embodiments, the milled grain which is used in the process has a particle size such that more than 50% of the material will fit through a sieve with a 0.5 mm mesh and in some embodiments more than 70% of the material will fit through a sieve with a 0.5 mm mesh (see, for example, WO2004/081193).

### Preparing a Slurry of Starch-Containing Material

The milled starch-containing material will be combined with water and recycled thin-stillage resulting in an aqueous slurry. The slurry will comprise between 15 to 55% ds w/w (e.g., 20 to 50%, 25 to 50%, 25 to 45%, 25 to 40%, and 20 to 35% ds). In some embodiments the recycled thin-stillage (backset) will be in the range of 10 to 70% v/v (e.g., 10 to 60%, 10 to 50%, 10 to 40%, 10 to 30%, 10 to 20%, 20 to 60%, 20 to 50%, 20 to 40% and also 20 to 30%).

Once the milled starch-containing material is combined with water and backset, the pH is not adjusted in the slurry. Further the pH is not adjusted after the addition of phytase and optionally alpha amylase to the slurry. In a preferred embodiment the pH of the slurry will be in the range of pH 4.5 to less than 6.0 (e.g., pH 4.5 to 5.8, pH 4.5 to 5.6, pH 4.8 to 5.8, pH 5.0 to 5.8, pH 5.0 to 5.4 and pH 5.2 to 5.5). The pH of the slurry may be between pH 4.5 and 5.2 depending on the amount of thin stillage added to the slurry and the type of material comprising the thin stillage. For example, the pH of the thin stillage may be between pH 3.8 and pH 4.5. As a further example Table 1 below illustrates the pH change that occurs with addition of increasing amounts of thin stillage to a whole ground corn slurry (32% ds) after stirring for 2 hours at 68.3° C.

TABLE 1

| Thin stillage w/w % | Final pH |
|---|---|
| 0 | 5.52 |
| 20 | 5.29 |
| 40 | 5.16 |
| 50 | 5.09 |
| 60 | 5.05 |
| 80 | 4.98 |
| 100 | 4.94 |

It should be mentioned, during ethanol production, acids can be added to lower the pH in the beer well to reduce the risk of microbial contamination prior to distillation.

In some embodiments, phytase will be added to the slurry. In other embodiments, in addition to the phytase, an alpha amylase will be added to the slurry. In some embodiments, the phytase and alpha amylase will be added to the slurry sequentially and in other embodiments the phytase and alpha amylase will be added simultaneously. In some embodiments, the slurry comprising the phytase and optionally the alpha amylase will be incubated (pretreated) for a period of 5 minutes to 8 hours (e.g., 5 minutes to 6 hours, 5 minutes to 4 hours 5 minutes to 2 hours, and 15 minutes to 4 hours). In other embodiments the slurry will be incubated at a temperature in the range of 40 to 115° C., (e.g. 45 to 80° C., 50 to 70° C., 50 to 75° C., 60 to 110° C., 60 to 95° C., 70 to 110° C., and 70 to 85° C.).

In other embodiments, the slurry will be incubated at a temperature of 0 to 30° C. (e.g. 0 to 25° C., 0 to 20° C., 0 to 15° C., 0 to 10° C. and 0 to 5° C.) below the starch gelatinization temperature of the starch-containing material. In some embodiments, the temperature will be below 68° C., below 65° C., below 62° C., below 60° C. and below 55° C. In some embodiments, the temperature will be above 45° C., above 50° C., above 55° C. and above 60° C. In some embodiments, the incubation of the slurry comprising a phytase and an alpha amylase at a temperature below the starch gelatinization temperature is referred to as a primary (1°) liquefaction.

In one embodiment the milled starch-containing material is corn or milo. The slurry comprises 25 to 40% ds, the pH is in the range of 4.8 to 5.2, and the slurry is incubated with a phytase and optionally an alpha amylase for 5 minutes to 2 hours, at a temperature range of 60 to 75° C.

Currently, it is believed that commercially available microbial alpha amylases used in the liquefaction process are not stable enough to produce liquefied starch substrate from a dry mill process using whole ground grain at a temperature above 80° C. at a pH level that is less than pH 5.6. The stability of many commercially available alpha amylases is reduced at a pH of less than about 4.0.

In a further liquefaction step, the incubated or pretreated starch-containing material will be exposed to an increase in temperature such as 0 to 45° C. above the starch gelatinization temperature of the starch-containing material. (e.g. 70° C. to 120° C., 70° C. to 110° C., and 70° C. to 90° C.) for a period of time of 2 minutes to 6 hours (e.g. 2 minutes to 4 hrs) at a pH of about 4.0 to 5.5 more preferably between 1 hour to 2 hours. The temperature can be increased by a conventional high temperature jet cooking system for a short period of time for example for 1 to 15 minutes. Then the starch maybe further hydrolyzed at a temperature ranging from 75° C. to 95° C., (e.g., 80° C. to 90° C. and 80° C. to 85° C.) for a period of 15 to 150 minutes (e.g., 30 to 120 minutes). In a preferred embodiment, the pH is not adjusted during these process steps and the pH of the liquefied mash is in the range of pH 4.0 to pH 5.8 (e.g., pH 4.5 to 5.8, pH 4.8 to 5.4, and pH 5.0 to 5.2). In some embodiments, a second dose of thermostable alpha amylase will be added to the secondary liquefaction step, but in other embodiments there will not be an additional dosage of alpha amylase.

The incubation and liquefaction steps according to the invention may be followed by saccharification and fermentation steps well known in the art.

### Saccharification and Fermentation

Liquefied starch-containing material is saccharified in the presence of saccharifying enzymes such as glucoamylases. The saccharification process may last for 12 hours to 120 hours (e.g. 12 to 90 hours, 12 to 60 hours and 12 to 48 hours). However, it is common to perform a pre-saccharification step for about 30 minutes to 2 hours (e.g., 30 to 90 minutes) in a temperature range of 30 to 65° C. and typically around 60° C. which is followed by a complete saccharification during fermentation referred to as simultaneous saccharification and fermentation (SSF).

Fermentable sugars, (e.g. dextrins, monosaccharides, particularly glucose) are produced from enzymatic saccarification. These fermentable sugars may be further purified and/or converted to useful sugar products. In addition the sugars may be used as a fermentation feedstock in a microbial fermentation process for producing end-products, such as alcohol (e.g., ethanol and butanol), organic acids (e.g., succinic acid and lactic acid), sugar alcohols (e.g., glycerol), ascorbic acid intermediates (e.g., gluconate, 2-keto-D-gluconate, 2,5-diketo-D-gluconate, and 2-keto-L-gulonic acid), amino acids (e.g., lysine), proteins (e.g., antibodies and fragment thereof).

In a preferred embodiment, the fermentable sugars obtained during the liquefaction process steps are used to produce alcohol and particularly ethanol. In ethanol production a SSF process is commonly used wherein the saccharifying enzymes and fermenting organisms (e.g., yeast) are added together and then carried out at a temperature of 30° C. to 40° C.

The organism used in fermentations will depend on the desired end-product. Typically if ethanol is the desired end product yeast will be used as the fermenting organism. In some preferred embodiments, the ethanol-producing microorganism is a yeast and specifically Saccharomyces such as strains of S. cerevisiae (U.S. Pat. No. 4,316,956). A variety of S. cerevisiae are commercially available and these include but are not limited to FALI (Fleischmann's Yeast), SUPERSTART (Alltech), FERMIOL (DSM Specialties), RED STAR (Lesaffre) and Angel alcohol yeast (Angel Yeast Company, China). The amount of starter yeast employed in the methods is an amount effective to produce a commercially significant amount of ethanol in a suitable amount of time, (e.g. to produce at least 10% ethanol from a substrate having between 25-40% DS in less than 72 hours). Yeast cells are generally supplied in amounts of 10⁴ to 10¹², and preferably from 10⁷ to 10¹⁰ viable yeast count per ml of fermentation broth. The fermentation will include in addition to a fermenting microorganisms (e.g. yeast), nutrients, optionally additional enzymes, including but not limited to phytases. The use of yeast in fermentation is well known and reference is made to THE ALCOHOL TEXTBOOK, K. JACQUES ET AL., EDS. 1999, NOTTINGHAM UNIVERSITY PRESS, UK.

In further embodiments, by use of appropriate fermenting microorganisms as known in the art, the fermentation end product may include without limitation glycerol, 1,3-propanediol, gluconate, 2-keto-D-gluconate, 2,5-diketo-D-gluconate, 2-keto-L-gulonic acid, succinic acid, lactic acid, amino acids and derivatives thereof. More specifically when lactic acid is the desired end product, a Lactobacillus sp. (L. casei) may be used; when glycerol or 1,3-propanediol are the desired end-products E. coli may be used; and when 2-keto-D-gluconate, 2,5-diketo-D-gluconate, and 2-keto-L-gulonic acid are the desired end products, Pantoea citrea may be used as the fermenting microorganism. The above enumerated list are only examples and one skilled in the art will be aware of a number of fermenting microorganisms that may be appropriately used to obtain a desired end product.

### Recovery

Optionally, following fermentation, alcohol (e.g. ethanol) may be extracted by for example distillation optionally followed by one or more process steps.

In some embodiments, the yield of ethanol produced by the methods encompassed by the invention will be at least 8%, at least 10%, at least 12%, at least 14%, at least 15%, at least 16%, at least 17% and at least 18% (v/v). and at least 23% v/v. The ethanol obtained according to processes of the invention may be used as a fuel ethanol, potable ethanol or industrial ethanol.

In further embodiments, the end product may include the fermentation co-products such as distillers dried grains (DDG) and distiller's dried grain plus solubles (DDGS), which for example may be used as an animal feed.

### Enzymes Used in the Process Steps Phytases—

Phytases useful for the invention include enzymes capable of hydrolyzing phytic acid under the defined conditions of the incubation and liquefaction steps. In some embodiments, the phytase is capable of liberating at least one inorganic phosphate from an inositol hexaphosphate (phytic acid). Phytases can be grouped according to their preference for a specific position of the phosphate ester group on the phytate molecule at which hydrolysis is initiated, (e.g., as 3-phytases (EC 3.1.3.8) or as 6-phytases (EC 3.1.3.26)). A typical example of phytase is myo-inositol-hexakiphosphate-3-phosphohydrolase.

Phytases can be obtained from microorganisms such as fungal and bacterial organisms. Some of these microorganisms include e.g. Aspergillus (e.g., A. niger, A. terreus, A. ficu and A. fumigatus), Myceliophthora (M. thermophila), Talaromyces (T. thermophilus) Trichoderma spp (T. reesei). and Thermomyces (WO 99/49740). Also phytases are available from Penicillium species, e.g., P. hordei (ATCC No. 22053), P. piceum (ATCC No. 10519), or P. brevicompactum (ATCC No. 48944). See, for example U.S. Pat. No. 6,475,762. In addition, phytases are available from Bacillus (e.g. B. subtilis, Pseudomonas, Peniophora, E. coli, Citrobacter, Enterbacter and Buttiauixella (see WO2006/043178).

Commercial phytases are available such as NATUPHOS (BASF), RONOZYME P (Novozymes A/S), PHZYME (Danisco A/S, Diversa) and FINASE (AB Enzymes). The method for determining microbial phytase activity and the definition of a phytase unit has been published by Engelen et al. (1994) J. of AOAC International, 77: 760-764. The phytase may be a wild-type phytase, a variant or fragment thereof.

In one embodiment, the phytase useful in the present invention is one derived from the bacterium Buttiauxiella spp. The Buttiauxiella spp. includes B. agrestis, B. brennerae, B. ferragutiase, B. gaviniae, B. izardii, B. noackiae, and B. warmboldiae. Strains of Buttiauxiella species are available from DSMZ, the German National Resource Center for Biological Material (Inhoffenstrabe 7B, 38124 Braunschweig, Germany). Buttiauxiella sp. strain P1-29 deposited under accession number NCIMB 41248 is an example of a particularly useful strain from which a phytase may be obtained and used according to the invention. In some embodiments, the phytase is BP-wild type, a variant thereof (such as BP-11) disclosed in WO 06/043178 or a variant as disclosed in U.S. patent application Ser. No. 11/714,487, filed Mar. 6, 2007 (published as US 2008-0220498). For example, a BP-wild type and variants thereof are disclosed in Table 1 of WO 06/043178, wherein the numbering is in reference to SEQ ID NO:3 of the published PCT application.

In one preferred embodiment, a phytase useful in the instant invention is one having at least 75%, at least 80%, at least 85%, at least 88%, at least 90%, at least 93%, at least 95%, at least 96%, at least 97%, at least 98% and at least 99% sequence identity to the amino acid sequence set forth in SEQ ID NO: 1 shown in Table 2 and variants thereof. More preferably, the phytase will have at least 95% to 99% sequence identity to the amino acid sequence set forth in SEQ ID NO: 1 or variants thereof. In some embodiments, the phytase comprises or consists of the amino acid sequence of SEQ ID NO:1.

TABLE 2

Mature protein sequence of Buttiauxiella
BP-17 phytase

(SEQ ID NO:1)

NDTPASGYQV EKVVILSRHG VRAPTKMTQT MRDVTPNTWP
EWPVKLGYIT PRGEHLISLM GGFYRQKFQQ QGILSQGSCP
TPNSIYVWAD VDQRTLKTGE AFLAGLAPQC GLTIHHQQNL
EKADPLFHPV KAGTCSMDKT QVQQAVEKEA QTPIDNLNQH
YIPFLALMNT TLNFSTSAWC QKHSADKSCD LGLSMPSKLS
IKDNGNKVAL DGAIGLSSTL AEIFLLEYAQ GMPQAAWGNI
HSEQEWASLL KLHNVQFDLM ARTPYIARHN GTPLLQAISN
ALNPNATESK LPDISPDNKI LFIAGHDTNI ANIAGMLNMR
WTLPGQPDNT PPGGALVFER LADKSGKQYV SVSMVYQTLE
QLRSQTPLSL NQPAGSVQLK IPGCNDQTAE GYCPLSTFTR
VVSQSVEPGC QLQ

In some embodiments the amount (dosage) of phytase used in the incubation and/or liquefaction processes is in the range of about 0.001 to 50 FTU/g ds, (e.g. in the range of about 0.01 to 25 FTU/g ds, about 0.01 to 15 FTU/g ds, about 0.01 to 10 FTU/g ds, about 0.05 to 15 FTU/g ds, and about 0.05 to 5.0 FTU/g.

### Alpha Amylases—

In some preferred embodiments, the alpha amylase is an acid stable alpha amylase which, when added in an effective amount, has activity in the pH range of 3.0 to 7.0 and preferably from 3.5 to 6.5. Alpha amylases useful according to the invention may be fungal alpha amylases or bacterial alpha amylases. Further, the alpha amylase may be a wild-type alpha amylase, a variant or fragment thereof or a hybrid alpha amylase which is derived from for example a catalytic domain from one microbial source and a starch binding domain from another microbial source.

In some embodiments, the process according to the invention is particularly useful with an alpha amylase which is not stable below a pH of 5.6 at a high temperature (e.g. greater than 85° C., or greater than 80° C.).

Examples of fungal alpha amylases include those obtained from filamentous fungal strains including but not limited to strains of Aspergillus sp. (e.g., A. niger, A. kawachi, and A. oryzae); Trichoderma sp., Rhizopus sp., Mucor sp., and Penicillium sp.

Examples of bacterial alpha amylases include those obtained from bacterial strains including but not limited to strains of: Bacillus sp., such as B. licheniformis, B. stearothermophilus, B. amyloliquefaciens, B. subtilis, B. lentus, and B. coagulans. Particularly, B. licheniform is, B. stearothermophilus and B. amyloliquefaciens. Preferably one of the bacterial alpha amylases used in the processes of the invention include one of the alpha amylases described in U.S. Pat. No. 5,093,257; U.S. Pat. No. 5,763,385; U.S. Pat. No. 5,824,532; U.S. Pat. No. 5,958,739; U.S. Pat. No. 6,008,026; U.S. Pat. No. 6,093,563; U.S. Pat. No. 6,187,576; U.S. Pat. No. 6,361,809; U.S. Pat. No. 6,867,031; US 2006/0014265; WO 96/23874, WO 96/39528; WO 97/141213, WO 99/19467; and WO 05/001064.

Commercially available alpha amylases compositions contemplated for use in the processes encompassed by the invention include: SPEZYME™ AA; SPEZYME™ FRED; SPZYME™ XTRA; GZYME™ 997; and CLARASE™ L (Danisco US Inc, Genencor Division); TERMAMYL™ 120-L, LC and SC and SUPRA (Novozymes Biotech); LIQUOZYME™ X, LIQUEZYME SC and SAN™SUPER (Novozymes A/S) and Fuelzyme™ LF (Diversa).

In some embodiments, the amount of alpha amylase useful in the processes of the invention is an effective amount of alpha amylase which is well known to a person of skill in the art for example 0.1 to 50 AAU/gds, (e.g., 0.1 to 25 AAU/gds, 0.5 to 15 AAU/gds, and preferably 1.0 to 10 AAU/gds).

The enzyme compositions useful in the processes encompassed by the invention may include blended or formulated enzyme compositions of any phytase and an alpha amylase and particularly a thermostable alpha amylase.

In some embodiments, the alpha amylase will include an alpha amylase derived from Bacillus stearothermophilus such as SPEZYME™ AA, SPEZYME™ FRED or SPEZYME™ XTRA.

In some embodiments, the useful enzyme compositions will include BP-WT or BP-17, SPEZYME™ XTRA and optionally SPEZYME™ FRED. In certain embodiments, the phytase may be combined with an alpha amylase such as TERMAMYL™ SC or SUPRA and Liquozyme SC.

In some embodiments, when a phytase composition and an alpha amylase composition are used in a process step according to the invention, the ratio of phytase (FTU/g ds) to alpha amylase (AAU/g ds) is from about 15:1 to 1:15. In other embodiments, the ratio of phytase to alpha amylase is from about 10:1 to 1:10, also 5:1 to 1:5, 3:1 to 1:3, 2:1 to 1:2, and 3:1 to 1:2.

Enzyme compositions comprising the phytase and alpha amylase either in a blended formulation or individually include starch conversion compositions for example MAXALIQ™One (Danisco US Inc, Genencor Division).

In some non-limiting embodiments, the enzyme blend or compositions will include: a) a BP-17 phytase having at least 95%, or at least 97% or at least 99% sequence identity to SEQ ID NO: 1 and a thermostable bacterial alpha amylase; b) an E. coli phytase (e.g., PHYZYME XP) and an acid stable alpha amylase and c) an Aspergillus niger phytase and a thermostable bacterial alpha amylase.

The process steps according to the invention may impart added value with respect to animal feeds because the addition of phytase in the incubation step, either simultaneously or sequentially with the addition of alpha amylase results in an increased thermostability and/or pH stability of the alpha amylase at a lower pH. It is well known that extra phytic acid, myo-inositol hexakis-phosphate, which is the primary storage form of phosphate in cereals/grains and oil seeds is only partly utilized by monogastric animals (e.g., poultry and pigs) and therefore it is an undesirable component of grain or cereals in feed formulations. Phytate is also known to bind essential minerals such as zinc, iron, calcium, magnesium and proteins resulting in a reduction in the bioavailability, and further it has been shown that phytate and other myo-inositol phosphate esters exhibit an alpha amylase inhibitory effect on the hydrolysis of starch. As a consequence, the use of microbial phytases in many feed formulations has long been established (e.g., Phyzyme™ XP 5000 from Danisco US Inc, Genencor Division, Finase™ from AB Enzymes, GODO PHY™ from Godo Shusei Japan; Allzyme™ Phytase from Altech; Natuphos™ from BASF; and Ronozyme™ P from DSM/Novozyme). However, by inclusion of phytase in the process steps according to the invention the co-products such as DDGS have an increased value.

### Saccharifying Enzymes

Glucoamylases (GA) (E.C. 3.2.1.3.) are used as saccharifying enzymes and these may be derived from the heterologous or endogenous protein expression of bacteria, plants and fungi sources. Preferred glucoamylases useful in the compositions and methods of the invention are produced by several strains of filamentous fungi and yeast. In particular, glucoamylases secreted from strains of Aspergillus and Trichoderma are commercially important.

Suitable glucoamylases include naturally occurring wild-type glucoamylases as well as variants and genetically engineered mutant glucoamylases (e.g. hybrid glucoamylases).

Glucoamylases are also obtained from strains of Aspergillus, (A. niger, See, Boel et al., (1984) EMBO J. 3:1097-1102; WO 92/00381 and U.S. Pat. No. 6,352,851); A. oryzae, See, Hata et al., (1991) Agric. Biol. Chem. 55:941-949 and A. shirousami, See, Chen et al., (1996) Prot. Eng. 9:499-505); strains of Talaromyces such as those derived from T. emersonii, T. leycettanus, T. duponti and T. thermophilus (WO 99/28488; U.S. Pat. No. RE: 32,153; U.S. Pat. No. 4,587,215); strains of Trichoderma, such as T. reesei and particularly glucoamylases having at least 80%, 85%, 90% and 95% sequence identity to SEQ ID NO: 4 disclosed in U.S. Pat. No. 7,413,887; strains of Rhizopus, such as R. niveus and R. oryzae; strains of Mucor and strains of Humicola, such as H. grisea (See, Boel et al., (1984) EMBO J. 3:1097-1102; WO 92/00381; WO 00/04136; Chen et al., (1996) Prot. Eng. 9:499-505; Taylor et al., (1978) Carbohydrate Res. 61:301-308; USP. 4,514,496; U.S. Pat. No. 4,092,434; U.S. Pat. No. 4,618,579; Jensen et al., (1988) Can. J. Microbiol. 34:218-223 and SEQ ID NO: 3 of WO 2005/052148). In some embodiments, the glucoamylase will have at least 85%, 90%, 92%, 94%, 95%, 96%, 97%, 98% and 99% sequence identity to the amino acid sequence of SEQ ID NO: 3 of WO 05/052148. Other glucoamylases useful in the present invention include those obtained from Athelia rolfsii and variants thereof (WO 04/111218).

Enzymes having glucoamylase activity used commercially are produced for example, from Aspergillus niger (trade name DISTILLASE, OPTIDEX L-400 and G ZYME G990 4X from Danisco US, Inc, Genencor Division.) or Rhizopus species (trade name CU.CONC from Shin Nihon Chemicals, Japan). Also the commercial digestive enzyme, trade name GLUCZYME from Amano Pharmaceuticals, Japan (Takahashi et al., (1985) J. Biochem. 98:663-671). Additional enzymes include three forms of glucoamylase (E.C.3.2.1.3) of a Rhizopus sp., namely “Gluc1” (MW 74,000), “Gluc2” (MW 58,600) and “Gluc3” (MW 61,400). Also the enzyme preparation GC480 (Danisco US, Inc, Genencor Division) finds use in the invention. The above mentioned glucoamylases and commercial enzymes are not intended to limit the invention but are provided as examples only.

### Secondary Enzymes—

While some embodiments of the invention include the use of enzyme compositions or blends of an alpha-amylase and a phytase, and further a glucoamylase, optionally other enzymes may be used in the process steps. For example, other enzyme useful during liquefaction include without limitation: cellulases, hemicellulases, xylanase, proteases, phytases, pullulanases, beta amylases lipases, cutinases, pectinases, beta-glucanases, galactosidases, esterases, cyclodextrin transglycosyltransferases (CGTases), beta-amylases and combinations thereof.

In some embodiments, an additional enzyme is a second alpha amylase such as a bacterial or fungal alpha amylase, and in other embodiments the alpha amylase is a derivative, mutant or variant of a fungal or bacterial alpha amylase. Non-limiting examples of an additional alpha amylases useful in the process includes the alpha amylase enumerated above including alpha amylases derived from strains of Bacillus, Aspergillus, Trichoderma, Rhizopus, Fusarium, Penicillium, Neurospora and Humicola.

Some preferred additional alpha amylases are derived from Bacillus including B. licheniformis, B. lentus, B. coagulans, B. amyloliquefaciens, B. stearothermophilus, B subtilis, and hybrids, mutants and variants thereof (U.S. Pat. No. 5,763,385; U.S. Pat. No. 5,824,532; U.S. Pat. No. 5,958,739; U.S. Pat. No. 6,008,026 and U.S. Pat. No. 6,361,809). Some of these amylases are commercially available e.g., TERMAMYL and SUPRA available from Novo Nordisk A/S, ULTRATHIN from Diversa, LIQUEZYME SC from Novo Nordisk A/S and SPEZYME FRED, SPEZYME XTRA and GZYME G997 available from Danisco US, Inc, Genencor Division.

In another embodiment, the invention may include the addition of a second phytase which may be the same or different from the phytase used in the incubation step. Any of the phytases discussed in the section herein on phytases can be used.

Cellulases may also be incorporated with the alpha amylase and glucoamylase. Cellulases are enzyme compositions that hydrolyze cellulose (β-1, 4-D-glucan linkages) and/or derivatives thereof, such as phosphoric acid swollen cellulose. Cellulases include the classification of exo-cellobiohydrolases (CBH), endoglucanases (EG) and β-glucosidases (BG) (EC3.2.191, EC3.2.1.4 and EC3.2.1.21). Examples of cellulases include cellulases from Penicillium, Trichoderma, Humicola, Fusarium, Thermomonospora, Cellulomonas, Clostridium and Aspergillus. Commercially available cellulases sold for feed applications are beta-glucanases such as ROVABIO (Adisseo), NATUGRAIN (BASF), MULTIFECT BGL (Danisco US, Inc, Genencor Division) and ECONASE (AB Enzymes).

Xylanases may also be included in the process steps. Xylanases (e.g. endo-β-xylanases (E.C. 3.2.1.8), which hydrolyze the xylan backbone chain may be from bacterial sources, such as Bacillus, Streptomyces, Clostridium, Acidothermus, Microtetrapsora or Thermonospora. In addition xylanases may be from fungal sources, such as Aspergillus, Trichoderma, Neurospora, Humicola, Penicillium or Fusarium. (See, for example, EP473 545; U.S. Pat. No. 5,612,055; WO 92/06209; and WO 97/20920). Commercial preparations include MULTIFECT and FEEDTREAT Y5 (Danisco US, Inc, Genencor Division), RONOZYME WX (Novozymes A/S) and NATUGRAIN WHEAT (BASF).

Proteases may also be included in the process steps. Proteases may be derived from Bacillus such as B. amyloliquefaciens, B. lentus, B. licheniformis, and B. subtilis. These sources include subtilisin such as a subtilisin obtainable from B. amyloliquefaciens and mutants thereof (U.S. Pat. No. 4,760,025). Suitable commercial protease includes MULTIFECT P 3000 (Danisco US, Inc., Genencor Division) and SUMIZYME FP (Shin Nihon). Proteases are also derived from fungal sources such as Trichoderma, Aspergillus, Humicola and Penicillium. In some preferred embodiments, acid fungal proteases may also be included in the process steps, for example, those obtained from Aspergillus, Trichoderma, Mucor and Rhizopus. In one embodiment, the acid fungal protease is an acid fungal protease as disclosed in WO 06/073839.

### EXPERIMENTAL

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

In the disclosure and experimental section which follows, the following abbreviations apply: wt % (weight percent); ° C. (degrees Centigrade); H₂O (water); dH₂O (deionized water); dIH₂O (deionized water, Milli-Q filtration); g or gm (grams); fg (micrograms); mg (milligrams); kg (kilograms); μl (microliters); mL and ml (milliliters); mm (millimeters); μm (micrometer); M (molar); mM (millimolar); μM (micromolar); U (units); MW (molecular weight); sec (seconds); min(s) (minute/minutes); hr(s) (hour/hours); DO (dissolved oxygen); W/V (weight to volume); W/W (weight to weight); v/v (volume to volume); IKA (IKA Works Inc. 2635 North Chase Parkway SE, Wilmington, N.C.); Genencor (Danisco US Inc, Genencor Division, Palo Alto, Calif.); Ncm (Newton centimeter) and ETOH (ethanol). eq (equivalents); N (Normal); ds or DS (dry solids content), SAPU (spectrophotometric acid protease unit, wherein in 1 SAPU is the amount of protease enzyme activity that liberates one micromole of tyrosine per minute from a casein substrate under conditions of the assay) and GAU (glucoamylase unit, which is defined as the amount of enzyme that will produce 1 g of reducing sugar calculated as glucose per hour from a soluble starch substrate at pH 4.2 and 60° C.).

### Methods

Viscosity Measurements: A glass cooker—viscometer, LR-2.5T system IKA was used to determine viscosity. In brief, the viscometer consists of a 2000 ml double walled glass vessel with an anchor mixer that is stirred by a Eurostar Labortechnik power control-viscometer (the viscosity range of the Viscoklick viscometer is 0-600 Ncm). In general for the examples described herein a slurry comprising starch containing material and an appropriate amount of enzyme was poured into the viscometer vessel. The temperature and viscosity were recorded during heating to 85° C. and incubation was continued for an additional 60 to 120 mins. Viscosity measured as Ncm was recorded at intervals.

The phytase used in some of the examples herein was Buttiauxiella phytase, BP-17 shown herein as SEQ ID NO: 1 (see also U.S. patent application Ser. No. 11/714,487, filed Mar. 6, 2007, incorporated by reference).

Carbohydrate Analysis by High Pressure Liquid Chromatographic (HPLC): The composition of the reaction products of oligosaccharides was measured by HPLC using a Beckman System Gold 32 Karat (Fullerton, Calif.) equipped with an HPLC column (Rezex 8 u8% H, Monosaccharides), maintained at 50° C. fitted with a refractive index (RI) detector (ERC-7515A, RI Detector (Anspec Company Inc.). Saccharides were separated based on molecular weight. A designation of DP1 is a monosaccharide, such as glucose; a designation of DP2 is a disaccharide, such as maltose; a designation of DP3 is a trisaccharide, such as maltotriose and the designation “DP4⁺” is an oligosaccharide having a degree of polymerization (DP) of 4 or greater.

Phytase Activity (FTU) is measured by the release of inorganic phosphate. The inorganic phosphate forms a yellow complex with acidic molybdate/vanadate reagent and the yellow complex is measured at a wavelength of 415 nm in a spectrophotometer and the released inorganic phosphate is quantified with a phosphate standard curve. One unit of phytase (FTU) is the amount of enzyme that releases 1 micromole of inorganic phosphate from phytate per minute under the reaction conditions given in the European Standard (CEN/TC 327,2005-TC327WI 003270XX)

Phytic acid content: Phytic acid was extracted from sample by adjusting the pH of the 5% slurry (if it is dry sample) to pH 10 and then determined by an HPLC method using an ion exchange column. Phytic acid was eluted from the column using a NaOH gradient system (Mike Pepsin for HPLC source) Phytic acid content in the liquid was then calculated by comparing to a phytic acid standard.

Alpha amylase activity (AAU) was determined by the rate of starch hydrolysis, as reflected in the rate of decrease of iodine-staining capacity measured spectrophotometrically. One AAU of bacterial alpha-amylase activity is the amount of enzyme required to hydrolyze 10 mg of starch per min under standardized conditions.

Alpha-amylase activity can also be determined as soluble starch unit (SSU) and is based on the degree of hydrolysis of soluble potato starch substrate (4% DS) by an aliquot of the enzyme sample at pH 4.5, 50° C. The reducing sugar content is measured using the DNS method as described in Miller, G. L. (1959) Anal. Chem. 31:426-428.

Glucoamylase Activity Units (GAU) were determined using the PNPG assay. The PNPG assay is based on the ability of glucoamylase enzyme to catalyze the hydrolysis of p-nitrophenyl-alpha-D-glucopyranoside (PNPG) to glucose and p-nitrophenol. At an alkaline pH the nitrophenol forms a yellow color that is measured spectrophotometrically at 400 nm used in the calculation for GAU. One Glucoamylase Unit is the amount of enzyme that will liberate one gram of reducing sugars calculated as glucose from a soluble starch substrate per hour under the specified conditions of the assay.

### Example 1

### Effect of Removal of Phytic Acid Inhibition on Alpha Amylase Thermostability

The effect of the removal of phytic acid inhibition on the increase in the thermostability of liquefying thermostable alpha amylase was studied in this example.

A slurry of whole ground corn (obtained from Badger State Ethanol, Monroe, Wis.) was mixed with water containing 50% v/v thin stillage to a final concentration of about 32% ds. Corn solids were prepared in a jacked kettle. The slurry was mixed well and the pH of the slurry was adjusted to pH 5.8, which is a typical pH of a liquefaction of a commercial ethanol process using sodium carbonate or sodium hydroxide. This slurry was mixed in a jacketed kettle and brought up to the pretreatment temperature of 65-70° C. Just prior to reaching 70° C., the liquefying enzymes SPEZYME™ Xtra (10 AAU per gram ds corn) or genetically modified alpha amylase from Bacillus stearothermophilus (SPEZYME™ Ethyl, from Danisco US Inc, Genencor Division) were added and a timer was started to begin the incubation or primary liquefaction step. The slurry was allowed to incubate for 40 minutes in the presence of the enzymes with or without added phytase (12 FTU per gram ds corn). The incubated slurry was then passed through a jet cooker (82-107° C.) which was preheated to the desired temperature using steam and water. The slurry was sent through the jet at maximum speed (1.5 setting) about 4 liters/minute. Using the first three loops of the hold coil resulted in a hold time of just over 3 minutes. After all of the water was displaced and the desired temperature held steady, an aliquot of solubilized corn mash was collected and placed in a secondary bath (overhead stirring) at 85° C. to begin the secondary liquefaction step (2° liquefaction). Samples were taken to test for viscosity (by Brookfield), brix and DE (by Schoorls) at 0, 30, 60 and 90 minutes. The results are summarized in Table 3.

TABLE 3

| | Phytase (BP-17) | | | |
|---|---|---|---|---|
| | in the incubation | | | Viscos- |
| Enzyme Treatment | (1° liquefaction) | Time @85° C. | DE | ity, CPS |
| SPEZYME ™ Xtra | No | 0 | 9.56 | 6840 |
| 10 AAU/gds | | 30 min | 9.41 | 9900 |
| pH 5.8 | | 60 min | 9.95 | 9880 |
| | | 90 min | 9.78 | 9800 |
| SPEZYME ™ Ethyl | No | 0 | 7.55 | 5060 |
| 10 AAU/gds | | 30 min | 7.88 | 4340 |
| pH 5.8 | | 60 min | 8.15 | 4240 |
| | | 90 min | 8.44 | 3750 |
| SPEZYME ™ Xtra | Yes | 8.27 | 11.04 | 1060 |
| 10 AAU/gds + | | 30 min | 15.73 | 700 |
| 12 FTU/gds BP-17 | | 60 min | 16.84 | 750 |
| pH 5.8 | | 90 min | 17.9 | 750 |

Addition of BP-17 phytase during incubation (primary liquefaction) reduced the phytic acid content of the whole ground corn from 0.60% ds corn to 0.09% ds corn (>85% reduction). It is also very clear from the data in Table 3 that the alpha amylases were inactivated at a jet cooking temperature of 107° C. based on DE development or viscosity reduction. However, the inclusion of phytase prior to jet cooking (which it is believed removed the phytic acid inhibition) resulted in a significant increase in the thermostability of the alpha amylases as shown by DE progression and viscosity reduction at 85° C. during the secondary liquefaction step.

### Example 2

### Effect of Removal of Phytic Acid Inhibition on Alpha Amylase pH Stability

The increase in the thermostability of alpha amylase due to the removal of the phytic acid inhibition of alpha amylase was further studied. The phytic acid was hydrolyzed using phytase prior to the secondary liquefaction of whole ground corn and the improvement in the pH stability at low pH was determined.

In a typical experiment, whole ground corn was slurried to a 32% (ds corn) by using a 50:50 ratio of water and thin stillage. The slurry pH was measured and found to be pH 5.15. The slurry was heated to 70° C. using water and steam in a jacketed kettle. The liquefaction enzymes, SPEZYME Xtra and BP-17 were added and the slurry was pretreated by holding the temperature at 70° C. for 40 minutes. After 40 minutes of pretreatment, the slurry was passed through a jet-cooker maintained at 107° C. with a 3 minutes hold time using a large pilot plant jet (equipped with an M103 hydro-heater). The liquefact was collected from the jet and placed in an 85° C. water bath. A second dose of alpha amylase was added to complete the hydrolysis.

The liquefact was continuously stirred and held at 85° C. for 90 minutes. Samples were collected at 0, 30, 60 and 90 minutes. All samples were tested for Brix, DE (using the Schoorls method), and for viscosity (Brookfield viscometer spindle 2 at 20 rpms). The liquefaction studies were also conducted using SPEZYME™ Ethyl and BP-17. The DE progression and viscosity data are summarized in Table 4.

Table 4 shows the DE Progression and viscosity reduction during liquefaction of whole ground corn without any pH adjustment.

TABLE 4

| | BP-17 −1° | | | |
|---|---|---|---|---|
| | liquefaction | | | |
| | step (40 min, | | | Viscos- |
| Enzyme Treatment | 70° C.) | Time@85° C., | DE | ity, CPS |
| SPEZYME ™ Xtra - | 12.8 FTU/gds | 0 | 10.38 | 3620 |
| 10 AAU/gds | | 30 min | 12.69 | 1630 |
| pH 5.15 | | 60 min | 14.69 | 1740 |
| | | 90 min | 15.62 | 2140 |
| SPEZYME ™ Ethyl | 12.8 FTU/gds | 0 | 8.38 | 2200 |
| 10 AAU/gds | | 30 min | 9.78 | 1280 |
| pH 5.15 | | 60 min | 11.70 | 1250 |
| | | 90 min | 12.54 | 1290 |

The results in Table 3 and Table 4 showed that the reduction of phytic acid inhibition of SPEZYME™ Xtra and SPEZYME™ Ethyl prior to the high temperature jet cooking at 107° C. of whole ground corn resulted in a significant increase in the low pH stability for activity as evidenced by a steady increase in the DE progression at 85° C. with a concomitant decrease in the viscosity of the liquefact. The data clearly showed that SPEZYME™ Xtra or SPEZYME™ Ethyl can be successfully used in the liquefaction process for whole ground corn at a pH 5.2 if the inhibition of the phytic acid is eliminated.

### Example 3

### Effect of the Use of Other Phytases on Alpha Amylase pH Stability

Commercially available microbial phytases such as Phyzyme™ XP 5000 from Danisco (E-Coli) and DSM Phytase L from DSM, The Netherlands (Aspergillus niger) were also tested in the primary liquefaction step for removing the phytic acid inhibition. Liquefaction trials were conducted as described in Example 1 using SPEZYME™ Xtra at 10 AAU/gds and phytase at 12 FTU/gds. The liquefact samples were taken to measure the residual phytic acid content. The DE progression at pH 5.2, viscosity reduction and the phytic acid reduction are shown in Table 5.

Table 5 shows a comparison of different commercially available phytases during liquefaction with no pH adjustment using whole ground corn.

TABLE 5

| | | % Phytic | | | |
|---|---|---|---|---|---|
| | | acid | | | |
| Enzyme Treatment | Phytase | removed | Time@85 C., | DE | Viscosity, CPS |
| SPEZYME Xtra | BP-17 | 97 | 0 | 10.38 | 3620 |
| 10 AAU/gds. | 12.0 FTU/gds | | 30 min | 12.69 | 1630 |
| pH 5.15 | corn | | 60 min | 14.69 | 1740 |
| | | | 90 min | 15.62 | 2140 |
| SPEZYME Ethyl | Phyzyme XP | 89 | 0 | 11.07 | 2440 |
| 10 AAU/gds. | 5000 E. coli) | | 30 min | 11.59 | 1710 |
| pH 5.15 | 12.0 FTU/gds | | 60 min | 12.33 | 1580 |
| | corn | | 90 min | 12.63 | 1710 |
| SPEZYME Ethyl | DSM Phytase | 95 | 0 | 11.34 | 1660 |
| 10 AAU/gds | L(Aspergillus | | 30 min | 11.63 | 1240 |
| pH 5.15 | niger) | | 60 min | 12.52 | 1180 |
| | 12.00 FTU/gds | | 90 min | 13.31 | 1560 |
| | corn | | | | |

The data in Table 5 showed that phytases from E. Coli (Phyzyme™ XP 5000) or Aspergillus niger (DSM Phytase™ L) did stabilize the SPEZYME™ Ethyl similarly to BP-17 Phytase (Buttiauxiella) when they were added under the primary liquefaction conditions of the whole ground corn slurry.

### Example 4

### Effect on Ethanol Production

Liquefacts were used as fermentation feedstocks in ethanol fermentation for alcohol production. The liquefact-1 (32% ds corn containing 50% thin stillage) from SPEZYME™ Xtra at pH 5.8 without phytase in the primary liquefaction step was used. Also used was the liquefact from Example 2 using SPEZYME™ Xtra with phytase treatment in the primary liquefaction step. The pH of the liquefact-1 was adjusted to 4.2 using dilute sulfuric acid as in the conventional ethanol process whereas the liquefact from Example 2 was used without any further pH adjustment. The liquefact from Example 2 was used as the no pH adjustment test for the process of the present invention. In each experiment tare weights of the vessels were obtained prior to preparation of media. A 32% DS corn ds liquefact (2 liters) was taken in a 2 L flask. Red Star Ethanol Red yeast (RED STAR (Lesaffre) inoculums were prepared by adding 10 grams of yeast and 1 gram of glucose to 40 grams of water under mild agitation for one hour. Five mls of each inoculum was added to equilibrated fermentors followed by the addition of G Zyme™ 480 Ethanol (Danisco US Inc, Genencor Division) at 0.4 GAU/gds.corn to initiate the simultaneous saccharification and fermentation. The initial gross weight was noted and the flask was placed in a water bath maintained at 32° C. The samples were taken at different intervals of time and analyzed for carbohydrate and ethanol content using HPLC. Fermentations were also carried out using one kilogram of each liquefact and weight loss during fermentation was measured at different intervals of time. Based on the weight loss due to loss of carbon dioxide, the alcohol was measured (Table 6). At the conclusion of the fermentation, a final gross weight was obtained. The broth was quantitatively transferred into a 5 L round bottom vessel. Distillation was performed under vacuum until approximately 800 mls of ethanol was collected in a receptacle containing 200 mls water. The ethanol was diluted to 2 L and was analyzed by HPLC. The weight and DS of the still bottoms was obtained prior to drying. Residual starch analysis was performed on the DDGS. Stoichiometric calculations were performed based on weight loss, distillation, and residual starch analysis.

Ethanol calculation using CO₂ weight loss:

Ethanol production (mmol)=CO₂loss(g)/88

Ethanol production (g)=(CO₂loss(g)/88)*92=>CO₂loss(g)*1.045

Ethanol production (ml)=((CO₂loss(g)/88)*92)/0.789=>CO₂loss(g)×1.325

TABLE 6
Comparison of DDGS from conventional liquefaction process from
the pH adjustment free process according to the invention.

| | | DDGS (% ds) | | | | |
|---|---|---|---|---|---|---|
| Liquefaction | Alcohol yield | | Phytic | % | Free | Sulfate |
| Conditions | (weight loss) | Starch | Acid | IP 6 | Phosphate | (mg/gds) |
| Conventional process | 2.70 | 7.25 | 0.6 | 100 | 1.20 | 1.92 |
| pH 5.8 | gallon/bushel | | | | | |
| pH Free Adjustment | 2.70 | 9.28 | 0.2 | 0 | 1.33 | 0.23 |
| Process pH 5.2 | gallon/bushel | | | | | |

The data in Table 6 shows major difference in free sulfate and phytic acid content between the conventional process and the no pH adjustment process according to the invention. Removal of phytic acid inhibition of thermostable alpha amylase in the incubation resulted in the DDGS with reduced phytic acid content, higher free available phosphate and reduced sulfate. Thus, the process with no pH adjustment confers pH stability at low pH for liquefying thermostable alpha amylases in the starch liquefaction.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of processing starch comprising a liquefaction step without a pH adjustment.

2. The method of claim 1, wherein the liquefaction step is performed at a pH range of about 4.5 to 5.4.

3. The method of claim 1 further comprising saccharifying the starch to fermentable sugars and producing alcohol from fermentable sugars without a pH adjustment.

4. A method of producing fermentable sugar from starch comprising the steps:
(a) mixing starch with water and thin stillage to obtain a slurry,
(b) treating the slurry with a phytase,
(c) liquefying the starch,
(d) adding an alpha-amylase, and
(e) saccharifying the liquefied starch to obtain fermentable sugars, wherein a pH adjustment is not performed during any of the steps (a), (b), (c), (d), or (e).

5. The method of claim 4, wherein the thin stillage is in a range of about 10 to 70% v/v.

6. The method of claim 4, wherein the slurry has a dry solids content of about 20 to 50% w/v.

7. The method of claim 4, wherein phytase is added either prior to or simultaneously with liquefying the starch.

8. The method of claim 4, wherein the phytase has at least 75% amino acid sequence identity to Buttiauxiella BP-17 phytase.

9. The method of claim 4, wherein the starch is liquefied at a pH range about 4.5 to 5.4.

10. The method of claim 4, wherein the alpha-amylase is added to the starch either during step (b) and/or simultaneously with liquefying the starch.

11. The method of claim 4 further comprising adding a second alpha-amylase dose during liquefying the starch.

12. The method of claim 4 further comprising purifying and/or isomerizing the fermentable sugars.

13. The method of claim 4 further comprising admixing a cellulase, a hemicellulase, a xylanase, a protease, a pullulanase, a beta-amylase, a lipase, a cutinase, a pectinase, a beta-glucanase, a galactosidase, an esterase, a cyclodextrin transglycosyltransferase, or any combination thereof during starch liquefaction.

14. The method of claim 4, wherein the starch is from corn, wheat, rye, barley, sorghum, and any combination thereof.

15. The method of claim 4, wherein the starch is from corn or corn mash.

16. A method of producing an alcohol from starch comprising the steps:
(a) mixing starch with water and thin stillage to obtain a slurry,
(b) treating the slurry with a phytase,
(c) liquefying the starch,
(d) adding an alpha-amylase,
(e) saccharifying the liquefied starch to obtain fermentable sugars, and
(f) fermenting the fermentable sugars using a fermenting microorganism to obtain an alcohol, wherein a pH adjustment is not performed during any of the steps (a), (b), (c), (d), (e), or (f).

17. The method of claim 16 wherein the saccharifying and fermenting steps are performed simultaneously.

18. The method of claim 16, wherein the alcohol is ethanol.


SEQUENCE LISTING (1 sequence)

SEQ ID NO: 1 - protein, 413 aa, Buttiauxiella spp.
  misc_feature: Mature protein sequence of Buttiauxiella BP-17 phytase
NDTPASGYQVEKVVILSRHGVRAPTKMTQTMRDVTPNTWPEWPVKLGYITPRGEHLISLMGGFYRQKFQQQGILSQGSCPTPNSIYVWADVDQRTLKTGEAFLAGLAPQCGLTIHHQQNLEKADPLFHPVKAGTCSMDKTQVQQAVEKEAQTPIDNLNQHYIPFLALMNTTLNFSTSAWCQKHSADKSCDLGLSMPSKLSIKDNGNKVALDGAIGLSSTLAEIFLLEYAQGMPQAAWGNIHSEQEWASLLKLHNVQFDLMARTPYIARHNGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ